(19) United States
(12) Patent Application Publication
(10) Pub. No.: US 2004/0043390 A1
(43) Pub. Date: Mar. 4, 2004

(54) Use of nucleotide sequences as carrier of cultural information

(75) Inventor: Beda M. Stadler, Bern (CH)

Correspondence Address: ROTHWELL, FIGG, ERNST & MANBECK, P.C., 1425 K STREET, N.W., SUITE 800, WASHINGTON, DC, 20005, US

(73) Assignee: ASAT AG Applied Science & Technology

(21) Appl. No.: 10/247,338

(22) Filed: Sep. 20, 2002

Related U.S. Application Data
(60) Provisional application No. 60/396,553, filed on Jul. 18, 2002.

(51) Int. Cl. C12Q 1/68; G06F 19/00; G01N 33/48; G01N 33/50; H04K 3/00

(52) U.S. Cl. 435/6; 702/20; 380/1

(57) ABSTRACT

Nucleotide sequences are used to store meaningful information, such as letters, words, phrases, signs, icons, musical notes, numbers or bits and bitmaps in any cultural context including languages, phonetics, multimedia applications, codes, abbreviations, personal and scientific information. The information is stored by creating a plurality of codons composed of nucleotides that it is readable by any technique that is capable of analyzing nucleotide sequences. The information can also be encrypted by all known or to be developed algorithms of cryptography.

This application claims the benefit of U.S. Provisional Application No. 60/298,376, filed Jul. 18, 2002.

### DESCRIPTION

Nucleotide sequences are used to store meaningful information, such as letters, words, phrases, signs, icons, musical notes, numbers or bits and bitmaps in any cultural context including languages, phonetics, multimedia applications, codes, abbreviations, personal and scientific information. The information is stored by creating a plurality of codons composed of nucleotides that it is readable by any technique that is capable of analyzing nucleotide sequences. The information can also be encrypted by all known or to be developed algorithms of cryptography.

Triplets of the nucleotides A, &, C and T represent the universal genetic code as it is used by most living organisms. This biological code is used to create the known amino acids and is an internationally accepted standard of denominating the triple code in the form of amino acid names, three-letter abbreviation or single letter abbreviations. The same meaningful DNA code naturally exists also as RNA, whereby the nucleotide Tymidine (T) is replaced by the nucleotide Uracil (U).

The meaning of the genetic code is shown in the following Table 1.

TABLE 1

Second Position of Codon

| | | | |
|---|---|---|---|
| T | C | A | G |

| | | | | | | | |
|---|---|---|---|---|---|---|---|
| First | T | TTT Phe [F] | TCT Ser [S] | TAT Tyr [Y] | TGT Cys [C] | T | Third |
| Position | | TTC Phe [F] | TCC Ser [S] | TAC Tyr [Y] | TGC Cys [C] | C | Position |
| | | TTA Leu [L] | TCA Ser [S] | TAA Ter [end] | TGA Ter [end] | A | |
| | | TTG Leu [L] | TCG Ser [S] | TAG Ter [end] | TGG Trp [W] | G | |
| | C | CTT Leu [L] | CCT Pro [P] | CAT His [H] | CGT Arg [R] | T | |
| | | CTC Leu [L] | CCC Pro [P] | CAC His [H] | CGC Arg [R] | C | |
| | | CTA Leu [L] | CCA Pro [P] | CAA Gln [Q] | CGA Arg [R] | A | |
| | | CTG Leu [L] | CCG Pro [P] | CAG Gln [Q] | CGG Arg [R] | G | |
| | A | ATT lle [I] | ACT Thr [T] | AAT Asn [N] | AGT Ser [S] | T | |
| | | ATC lle [I] | ACC Thr [T] | AAC Asn [N] | AGC Ser ]S] | C | |
| | | ATA lle [I] | ACA Thr [T] | AAA Lys [K] | AGA Arg [R] | A | |
| | | ATG Met [M] | ACG Thr [T] | AAG Lys [K] | AGG Arg [R] | G | |
| | G | GTT Val [V] | GCT Ala [A] | GAT Asp [D] | GGT Gly [G] | T | |
| | | GTC Val [V] | GCC Ala [A] | GAC Asp [D] | GGC Gly [G] | C | |
| | | GTA Val [V] | GCA Ala [A] | GAA Glu [E] | GGA Gly [G] | A | |
| | | GTG Val [V] | GCG Ala [A] | GAG Glu [E] | GGG Gly [G] | G | |

The present invention is based on the finding that nucleic acid molecules can be used to store meaningful information, which is different from the genetic code. The 4 nucleotides of DNA may be used in any combination and in any number of repeats, e.g. as a simple four-bit-storage (corresponding to the nucleotides A,C,G,T); as duplicates (4 times 4), creating a 16-bit code or similar to the universal genetic code as a triplet code (4×4×4=64) (see table below), creating 64 possibilities for information units etc.

Invented meaningful codes can be synthesized in the form of nucleotide sequences (DNA or RNA) and inserted or added to living and non-living systems. The retrieval of the sequences is made possible by nucleic acid detection methods, e.g. by sequencing or sequencing preceded by standard polymerase chain reaction (PCR) techniques whereby the primers may be part of the meaningful information. Synthesis by commercial DNA synthesizers is sufficient for most applications needing only trace amounts of DNA. Large scale production of meaningful DNA can be obtained through prokaryotic plasmids or eukaryotic vectors enabling also the production of much longer DNA.

Thus, a subject matter of the present invention is the use of a nucleic acid molecule as a carrier for information different from the genetic code, wherein said nucleic acid molecule comprises a plurality of codons, each comprising at least one nucleotide and wherein a codon corresponds to a specific meaning, i.e. an information unit, which is different from the meaning “amino acid” or “termination codon”.

A single codon may comprise at least one nucleotide, e.g. 1, 2, 3, 4, 5, 6 or more nucleotides. The codon length may be constant within the nucleic acid molecule or It may vary within the nucleic acid molecule, e.g. according to a predetermined algorithm

The specific meaning of a codon may be selected from letters, numbers, words, phrases, signs, icons, graphics, musical notes, colors, bits, bit maps and any combination thereof. The codon sequence is selected such that it contains information, which is composed of the meanings of a plurality of single codons.

The nucleic acid molecule is preferably selected from double-stranded or single-stranded DNA. Alternatively, the nucleic acid may also be RNA or a nucleic acid analogue comprising modified, i.e. non-naturally occurring nucleotides. The nucleic acid molecule is preferably produced by chemical synthesis, or by recombinant methods, including transcription, reverse transcription, replication, amplification, propagation in suitable host cells or host organisms, or any combination thereof. More preferably, the nucleic acid molecule is at least partially chemically synthesized. Furthermore, it is preferred that the nucleic acid molecule is biologically non-functional, i.e. it does not contain any meaningful information within the context of the genetic code, which particularly means that the nucleic acid molecule does not encode a biologically functional polypeptide or contain a regulatory sequence.

Furthermore, it is preferred that the nucleic acid molecule additionally comprises at least one identification segment, which does not necessarily comprise any information-carrying codons. Usually, the identification segment is suitable for hybridizing with a complementary probe sequence Alternatively, the identification segment may specifically bind to a protein, e.g. an antibody or a DNA-binding protein, such as a zinc finger domain, a leucin zipper domain, a DNA-binding repressor etc. In an especially preferred embodiment a nucleic acid molecule comprises at least two identification segments suitable for hybridizing with nucleic acid amplification primers and allowing amplification of the encoded sequence, e.g. by PCR.

The nucleic acids may be used for the labelling of objects or living organisms. The information may be encrypted or not.

The nucleic acid molecule may be applied as liquid formulation to objects, e.g. by spraying pipetting, immersing, pouring etc. Alternatively, the nucleic acid molecule may be embedded, e.g. as dehydrated molecule, into solid objects, such as metals, resins etc. For the labelling of living organisms usual DNA transfection techniques may be used.

In the following several preferred applications of the invention are explained in more detail:

Storing of Public or Secret Information

Products or organisms containing such additional meaningful nucleotide information can be labeled publically and open declaring the necessary PCR primers so that everybody may regain the same information from the product or the organism by sequencing and knowing the respective code. On the other hand, nucleotide sequences can be added to products or organisms secretly so that only the producer could regain the same information.

For example, a tiny amount of encoded and even encrypted meaningful information added as DNA to an orange juice could practically not be found by anybody in reasonable times without knowing the corresponding sequence as orange juice contains immensely more DNA from the orange and from organisms that were in contact during production. The information would represent actually a steganogram like nature and even if Its presence is suspected it would be almost impossible to be detected by an uninformed individuum.

Signatures and Propriety Declarations

Any product or living organism could be modified in a way that accessible or secret meaningful information is contained therein by a nucleotide sequence. For example, an ink producer may want to add a tiny amount of DNA to personalized ink, containing personal information (text, a logo, an image, etc., and all encrypted) of the Ink owner. This would give a signature and additional level of security.

A typical use would be the addition of a small amount of meaningful DNA into luxury articles, e.g. into perfumes for copyright protection. Resulting in an almost total security the same or a connected code could be spotted or sprayed onto porous packaging material. The canvas back of famous paintings could be sprayed with DNA to proof ownership and to make copying impossible.

Food producers may add DNA sequences to their products using publicly accessible codes or secret codes in order to resolve liability questions. Added on DNA sequences are an add on value, as DNA by itself is neither toxic nor dangerous but only represents a nutritional value. There is no need to label the product as GMO as the necessary quantities are almost 1000 times less then the regulatory levels for declaration.

Historical Information and Stability of Storage

It may be of interest to individuals, groups, societies or governments to record information for historical proof or mere documentation,

Non-living or living organisms may contain meaningful text, e.g. grass could be modified to contain the last will of the grass owner planted as a lawn in the back yard.

Any other form of text, picture, music or multimedia information could, of course, also be stored using nucleotides as it has been proven that this storage carriers can endure millions of years, a proof that for many other storage carriers has not yet been delivered (e.g. paper, magnetic tapes, CD-rom, etc.) Thus, information storage within nucleotide sequences is at presently the best documented form of keeping valuable information. Furthermore, the information, if associated with living organisms, can basically definitely be further propagated and renewed.

Traceability and Quality Control

The consumers wish for complete traceability could easily be fulfilled with labelling products or living systems with meaningful DNA Information. Even better than the today traceability of genetically modified foods, which contain genetic information that already exists in nature, new meaningful codes will also be readily re-recognized as either being degenerated, modified or altered in any way. Such a total traceability offers also a genetic marking for copyrights by putting genetically meaningful information in the vicinity of promoters that enduce a high rate of mutation. Thereby it could be proven that a given organism had been further propagated without explicit permission from the producer. On the other hand, inserted information can be protected from the effects of natural mutation by methods that are used in data communication or by repeating the same information several times in the same organisms.

If consumers wish they may take a sample e.g. from a meat meal in a restaurant and have it analyzed. If it contains a code that is described by regulatory agencies or by the producer they might trace their meat back to the breeding parents. Thus, regulatory agencies may ask for genetic stamping, so that ownership and liability are no more a matter of dispute.

An other example may be explosives containing an precise and batchwise DNA information to trace ammunition and other explosive containing weapons.

Environment Monitoring

It may be of public interest to voluntarily or involuntarily label products or living organisms. For example, it could even be of interest to NGO organizations to involuntarily mark oil freighters with encoded meaningful genetic material to prevent pollution in international waters. On the other hand, responsible industries may voluntarily label products with an environmental risk by genetic stamps to gain public goodwill and to avoid liability suits.

Secrete and Privileged Forms of Communication

It is clear that the technology of storing genetically meaningful information is of interest to exploit this technology in order to extend cryptographic and steganographic possibilities in combination with the technology. A simple cheese burger could become an information delivery system hard to crack as the information could reside within the sesame seeds, the weed, the meat, the cucumbre, the ketchup, the cheese, the spices or the contaminating bacteria.

Examples of Meaningful Codes

Below is Table 2 using the universal genetic code based on triplets (rows 1-3 of table) to invent new meaningful information codes.

[image]

Row 2. The examples in row 2 indicate the scientific 3-letter codes for the respective amino acids encoded by the triplets. The shown 3-letter combinations are not intended to be patented as they are generally used by the scientific community, but they are an example that any combinations of letters in any length could be associated with a given 3-letter codon. These letters may contain meaningful information, like in the case of the triplet TAA, representing a stop-codon or a termination signal.

Row 3. This row contains abbreviated information, a single or multiple letters, each pointing to a larger idea or concept or any product Again, the indicated letters are those that are presently used in science and cannot be patented, however, In any other meaning not pointing to the specific amino acids.

Rows 4-10 represent examples for other types of invented codes to transport information.

Row 4 is a very simple code composed of small and capital letters, numbers, space and a simple interpunctuation, In this simplest form the genetic code could be used to store plain text and numbers separated by spaces and points, but without additional interpunctuations.

Row 5 is an example of using the genetic code to store Iconographic information as it Is used today or as used in ancient languages such as hieroglyphs in the Egyptian language.

Row 6 is an example for storing information to provide directions, mathematical or physical symbols pointing to very complex communicative matters.

Row 7 is the Greek alphabet exemplifying that any language whether It had once existed, exists today, or will newly be invented, can be communicated using such a simple code.

Row 8 is an example that cultural concepts, such as symbols for planets or birth decades, star signs, smileys, skulls, crosses, other religious signs, ect. could be associated with the genetic code and thereby even transmitting information that is not universally understood as a single, defined concept to.

Row 9 would be a further development of a simple code as described in row 4, where a modifying triplet, e.g. GCA, would render in front of any other triplet a given capital letter into a small type letter, thus, extending a 64-letter code basically to a 128-sign code.

Row 10 is a further development and shows basically the typewriter layout as used today on computer keyboards, where several modifying triplets, here e.g. AGT, representing the shift key, AGC, representing the control key (CTRL) or AGA, representing the alternative graphics key (Alt G4r) Additionally any other modifying triplet could be defined extending the number of signs or letters to a great number, By doing so, it would be feasable e.g to encode thousands of Chinese letters,

Lane 10 is a further example that triplets can be left undefined or used redundantly in case size or meaning of the code asks for it.

Rows 11-14 are examples based on the ASCII code.

In row 11 is the internationally defined character and in rows 12-14 its corresponding decimal, octesimal or hexadecimal code. Thus, rows 12-13 are examples for codes that are based only on numerals. All numerical codes, such as the Roman numbering system, or other non-decimal systems and, of course, binary systems could be associated with the genetic code.

Row 14 is an example of combinatorial codes, whereby numerals and letters are used. Many industrial codes are basically also of the same type, e.g. the European norm codes (EN).

Random and Combinatorial Codes

The simple codes as depicted in row 2-14 can, of course, be randomized in any way, e.g. within one row or amongst information contained in the different examples in the different rows creating mixed codes.

Other Non-Illustrated Examples

Other forms of communication can also easily be stored within a single, duplicate, triplicate, quadruplicate or multiple nucleotide codon code, e.g bit maps, such as bit maps as in grafic files (e.g. GIF, JPEG, Tif. etc.) in order to generate images or other grafical information. However, for data intense DNA-storage such as bitmaps, duplicate codons will be more economic. Thereby 16 gray shades or colors could be stored directly in grafic files.

Musical notes and musical instructions could also be associated with nucleotide combinations to store music and sound, thereby it would even become possible to combine images and sounds, thus, storing information similar to video signals or other multi media applications.

Cryptographic Modification of Codes

Simple cryptographic modifications of the codes can be achieved by changing sequence of information or applying modern cryptographic algorithms based on existing or future algorithms. The most simplest form would be the storage of the Morse alphabet, barcodes, naval codes, etc.

1. A method for labeling objects or non-human organisms, comprising applying a nucleic acid molecule to said object or non-human organism, wherein said nucleic acid molecule carries information different from the genetic code and comprises a plurality of condons, each comprising at least one nucleotide and wherein a codon corresponds to a specific meaning.

2. The method of claim 1, wherein a codon comprises 1, 2, 3, 4, 5 or 6 nucleotides.

3. The method of claim 1, wherein the codon length is constant within the nucleic acid molecule.

4. The method of claim 1, wherein the condon length is variable within the nucleic acid molecule.

5. The method of claim 1, wherein a codon corresponds to a specific meaning selected from letters, numbers, words, phrases, signs, icons, musical notes, bits, bit maps and any combination thereof.

6. The method of claim 1, wherein the nucleic acid molecule is selected from double-stranded or single-stranded DNA or RNA.

7. The method of claim 1, wherein the nucleic acid molecule is at least partially chemically synthesized.

8. The method of claim 1, wherein the nucleic acid molecule is biologically non-functional.

9. The method of claim 1, wherein the codon meaning is encrypted.

10. The method of claim 1, wherein the nucleic acid molecule additionally comprises at least one identification segment.

11. The method of claim 10, wherein the identification segment is suitable for hybridizing with or binding to a probe sequence.

12. The method of claim 10, wherein the nucleic acid molecule comprises at least two identification segments suitable for hybridizing with nucleic acid amplification primers.

13. The method of claim 1 for labeling of objects.

14. The method of claim 13, wherein the objects are selected from foodstuffs, paper, clothes, and luxury articles.

15. The method of claim 1 for the labeling of non-human organisms.

16. The method of claim 15, wherein the organisms are selected from transgenic microorganisms, animals and plants.

17. The method of claim 1, wherein the nucleic acid molecule contains meaningful information composed of the meanings of a plurality of codons.
